(19) United States
(12) Patent Application Publication
(10) Pub. No.: US 2025/0131329 A1
(43) Pub. Date: Apr. 24, 2025

(54) METHOD OF GENERATING TRAINING DATA, METHOD OF GENERATING PREDICTION MODEL, COMPUTING DEVICE, AND NON-TRANSITORY COMPUTER-READABLE MEDIUM

(71) Applicant: NIHON KOHDEN CORPORATION, Tokyo (JP)

(72) Inventors: Takuya KAWASHIMA, Tokorozawa-shi (JP); Daisuke HORIGUCHI, Tokorozawa-shi (JP)

(73) Assignee: NIHON KOHDEN CORPORATION, Tokyo (JP)

(21) Appl. No.: 18/828,431

(22) Filed: Sep. 9, 2024

(30) Foreign Application Priority Data
Oct. 18, 2023 (JP) 2023-179590

(51) Int. Cl. G06N 20/00; G16H 50/30

(52) U.S. Cl. CPC G06N 20/00; G16H 50/30

(57) ABSTRACT

Training data is used in machine learning of a prediction model adapted to predict physiological information. An original data set includes a first observed value of an observed parameter for obtaining the physiological information acquired from a living body at a first time point, and a second observed value of the observed parameter acquired from the living body at a second time point different from the first time point. A first interpolation data set is generated by interpolating, with a first method, at least one value of the observed parameter in a time period between the first time point and the second time point. A second interpolation data set is generated by interpolating, with a second method different from the first method, at least one value of the observed parameter in the time period. The training data is generated so as to include the first and second interpolation data sets.

### CROSS-REFERENCE TO RELATED APPLICATION

The present application is based on Japanese Patent Application No. 2023-179590 filed on Oct. 18, 2023, the entire contents of which are incorporated herein by reference.

### BACKGROUND

The presently disclosed subject matter relates to a method performed by a computing device for generating training data that are to be used in machine learning of a prediction model adapted to predict physiological information. The presently disclosed subject matter also relates to a method of generating the prediction model, the computing device, and a non-transitory computer-readable medium having stored a computer program adapted to be executed by a processor installed in the computing device.

Prediction models that are generated with machine learning techniques to replace or support clinical decisions of medical workers in the field of medical clinical practice are known. For example, Non-patent Document 1 discloses a model adapted to predict specific physiological information based on time series data of an observed parameter acquired from a living body. Examples of specific physiological information include the future value of the observed parameter (used as a time series forecasting model), the in-hospital mortality probability (used as a classification model), and the remaining stay time of the intensive care unit (ICU) (used as a regression model). The Non-patent Document 1 reports that the prediction model exhibits excellent performance with respect to an observed parameter such as a heart rate that would be frequently acquired, whereas the prediction model does not exhibit good performance with respect to an observed parameter such as the number of leukocytes that would be intermittently acquired (difficult to acquire a sufficient amount of time series data).
Non-patent Document 1: G. Harerimana, et al. “A Multi-Headed Transformer Approach for Predicting the Patient's Clinical Time-Series Variables From Charted Vital Signs”, IEEE Access (Volume 10), 105993-106004, Oct. 3, 2022

### SUMMARY

It is demanded to suppress degradation of prediction accuracy of a prediction model adapted to predict physiological information even in an environment in which it is difficult to acquire a sufficient amount of time series data of an observed parameter.

An illustrative aspect of the presently disclosed subject matter provides a method of generating, with a computing device, training data to be used in machine learning of a prediction model adapted to predict physiological information, comprising:
receiving an original data set including a first observed value of an observed parameter for obtaining the physiological information that is acquired from a living body at a first time point, and a second observed value of the observed parameter that is acquired from the living body at a second time point that is different from the first time point;
generating a first interpolation data set by interpolating, with a first method, at least one value of the observed parameter in a time period between the first time point and the second time point;
generating a second interpolation data set by interpolating, with a second method that is different from the first method, at least one value of the observed parameter in the time period;
and generating the training data so as to include the first interpolation data set and the second interpolation data set.

An illustrative aspect of the presently disclosed subject matter provides a computing device configured to generate training data to be used in machine learning of a prediction model adapted to predict physiological information, comprising:
an interface configured to receive an original data set including a first observed value of an observed parameter for obtaining the physiological information that is acquired from a living body at a first time point, and a second observed value of the observed parameter that is acquired from the living body at a second time point that is different from the first time point; and
a processor configured to generate the training data so as to include a first interpolation data set and a second interpolation data set,
wherein the first interpolation data set is generated by interpolating, with a first method, at least one value of the observed parameter in a time period; and
wherein the second interpolation data set is generated by interpolating, with a second method that is different from the first method, at least one value of the observed parameter in the time period between the first time point and the second time point.

An illustrative aspect of the presently disclosed subject matter provides a non-transitory computer-readable medium having stored a computer program adapted to be executed by a processor installed in a computing device, the computer program being configured to cause, when executed, the computing device to:
receive an original data set including a first observed value of an observed parameter for obtaining the physiological information that is acquired from a living body at a first time point, and a second observed value of the observed parameter that is acquired from the living body at a second time point that is different from the first time point;
generate a first interpolation data set by interpolating, with a first method, at least one value of the observed parameter in a time period between the first time point and the second time point;
generate a second interpolation data set by interpolating, with a second method that is different from the first method, at least one value of the observed parameter in the time period; and
generate the training data so as to include the first interpolation data set and the second interpolation data set.

In general, the larger the amount of data included in the training data (the number of observed values), the more the capability of suppressing degradation of accuracy of the prediction performed by the prediction model to be generated. On the other hand, for example, the axillary temperature is generally measured at a frequency of once every several hours in a clinical practice, and there may be a case where it is difficult to secure time series data including a sufficient number of observed values with respect to the observed parameter. However, according to the configuration of each of the above illustrative aspects, the amount of data included in the training data through the interpolation processing can be augmented even in the case where the number of observed values included in the original data set is small. In addition, since the training data is generated so as to include multiple interpolation data sets generated with different interpolation methods, it is possible to suppress a bias in the augmentation tendency that may be caused by relying on a specific interpolation method. Accordingly, it is possible to suppress degradation of prediction accuracy of a prediction model adapted to predict physiological information even in an environment in which it is difficult to acquire a sufficient amount of time series data of an observed parameter.

An illustrative aspect of the presently disclosed subject matter provides a method of generating, with a computing device, the prediction model with the training data generated by the above method, comprising:
performing supervised machine learning such that an observed value of the observed parameter that is acquired after the second time point is regarded as a ground truth; and
configuring the prediction model so as to predict, with respect to multiple observed values of the observed parameter acquired at different time points as an input, an unobserved value of the observed parameter as the physiological information.

An illustrative aspect of the presently disclosed subject matter provides a method of generating, with a computing device, the prediction model with the training data generated by the above method, comprising:
performing supervised machine learning such that whether an event related to the observed parameter occurred at or after the second time point is regarded as a ground truth; and
configuring the prediction model so as to predict, with respect to multiple observed values of the observed parameter acquired at different time points as an input, a probability of occurrence of the event as the physiological information.

An illustrative aspect of the presently disclosed subject matter provides a method of generating, with a computing device, the prediction model with the training data generated by the above method, comprising:
performing supervised machine learning such that an observed value of a different observed parameter from the observed parameter that is acquired after the second time point is regarded as a ground truth; and
configuring the prediction model so as to predict, with respect to multiple observed values of the observed parameter acquired at different time points as an input, an observed value of the different observed parameter as the physiological information.

### DESCRIPTION OF THE DRAWINGS

FIG. 1 illustrates a functional configuration of a prediction system according to one exemplary embodiment.

FIG. 2 illustrates an exemplary flow of processing executed in a prediction device of FIG. 1.

FIG. 3 illustrates an exemplary processing for changing a weighting factor in FIG. 2.

FIG. 4 illustrates another exemplary processing for changing the weighting factor in FIG. 2.

FIG. 5 illustrates an exemplary flow of processing executed in the prediction system of FIG. 1.

FIG. 6 illustrates an exemplary flow of processing executed in the prediction system of FIG. 1.

### DESCRIPTION OF EMBODIMENTS

Exemplary embodiments will be described in detail with reference to the accompanying drawings. In the drawings, the scale is appropriately changed in order to make each element to be described have a recognizable size.

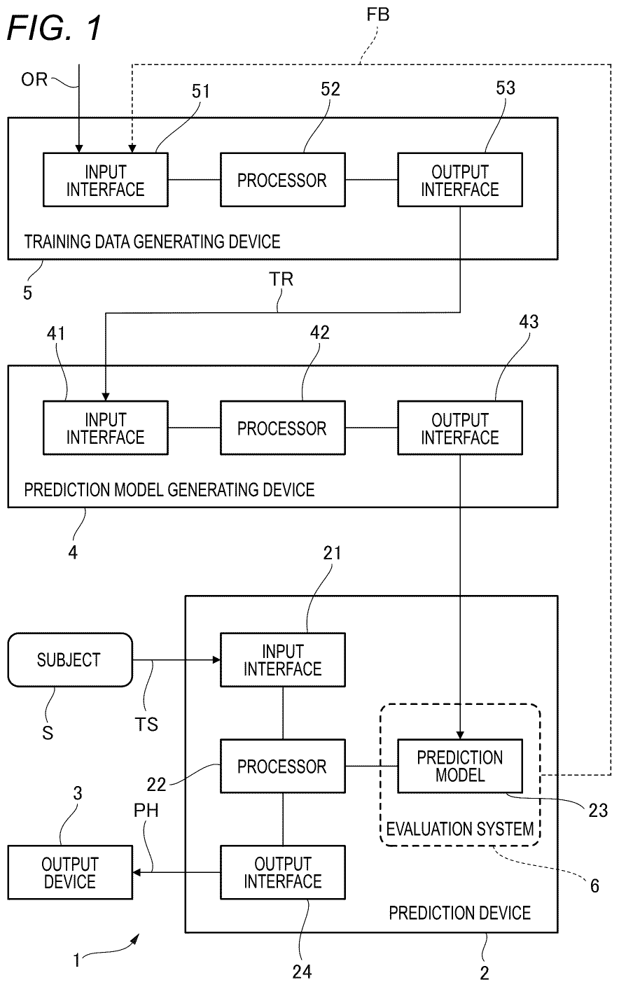

FIG. 1 illustrates a functional configuration of a prediction system 1 according to an exemplary embodiment. The prediction system 1 includes a prediction device 2.

The prediction device 2 according to the present example is a device configured to predict, based on time series data TS of axillary temperature acquired from a subject S at or before a certain time point, a value of the axillary temperature after the time point (in the future). The axillary temperature is an example of an observed parameter. Examples of other observed parameters include a respiratory rate that is obtained by visually observing thoracic motions, a Glasgow Coma Scale (GCS) that is obtained by visually confirming the state of consciousness, a urine volume that is measured in a urine collection bag, an index value that is obtained through a blood test (the number of erythrocytes, the number of leukocytes, a blood lactate value, an arterial oxygen partial pressure, and the like), and a blood pressure that is obtained by non-invasive measurement. A future value of the observed parameter is an example of the physiological information.

As used herein, the term “time series of an observed parameter” means changes over time of a value of an observed parameter that is acquired at multiple time points. The interval between the multiple time points may be constant or may not be constant.

The prediction device 2 includes an input interface 21. The input interface 21 is configured as a hardware interface that can receive the above-described time series data TS. The time series data TS may be inputted from an adequate sensor or measurement device attached to the subject S, or may be inputted from an adequate input device and a user operation. The time series data TS may be in the form of analog data or digital data, in accordance with the specification of the input source. In the case where the time series data TS is in the form of analog data, the input interface 21 includes an adequate conversion circuit including an A/D converter.

The prediction device 2 includes a processor 22 and a prediction model 23. The prediction model 23 is a computer algorithm adapted to be executed by the processor 22. The prediction model 23 is configured to output, as a prediction result, a forecasted value of the axillary temperature of the subject S in the future, with respect to the time series data TS as an input.

The prediction device 2 includes an output interface 24. The processor 22 is configured to input the time series data TS received by the input interface 21 to the prediction model 23, and configured to output, from the output interface 24, physiological information data PH corresponding to the prediction result outputted from the prediction model 23.

The prediction system 1 includes an output device 3. The output device 3 is configured to enable the physiological information data PH to be recognized by the user. Examples of the output device 3 include a display device, a printing device, an audio output device, a vibration generator, and a data transmission device. Accordingly, the user can recognize the predicted value of the axillary temperature of the subject S in the future, through the output device 3.

The output interface 24 of the prediction device 2 is configured as a hardware interface adapted to output the physiological information data PH. The physiological information data PH may be in the form of analog data or digital data, in accordance with the specification of the output device 3. In the case where the physiological information data PH is in the form of analog data, the output interface 24 includes an adequate conversion circuit including a D/A converter.

The prediction system 1 includes a prediction model generating device 4 and a training data generating device 5. The prediction model 23 of the prediction device 2 is generated through machine learning that is performed by the prediction model generating device 4. The training data generating device 5 generates training data TR that is used in the machine learning of the prediction model 23. The details of the machine learning will be described later. Each of the prediction model generating device 4 and the training data generating device 5 is an example of a computing device.

The training data generating device 5 includes an input interface 51. The input interface 51 is configured as a hardware interface adapted to receive original data set OR. The original data set OR according to the present example includes data corresponding to a time series of the axillary temperature acquired from the living body. In other words, the original data set OR includes data corresponding to multiple observed values of the axillary temperature acquired from the living body at multiple time points.

The original data set OR may be inputted from an adequate sensor or measurement device attached to the living body, or may be inputted from an adequate input device and a user operation. The original data set OR may be in the form of analog data or digital data, in accordance with the specification of the input source. In the case where the original data set OR is in the form of analog data, the input interface 51 includes an adequate conversion circuit including an A/D converter.

The training data generating device 5 includes a processor 52 and an output interface 53. The processor 52 is configured to generate training data TR based on the original data set OR, and output the training data TR from the output interface 53.

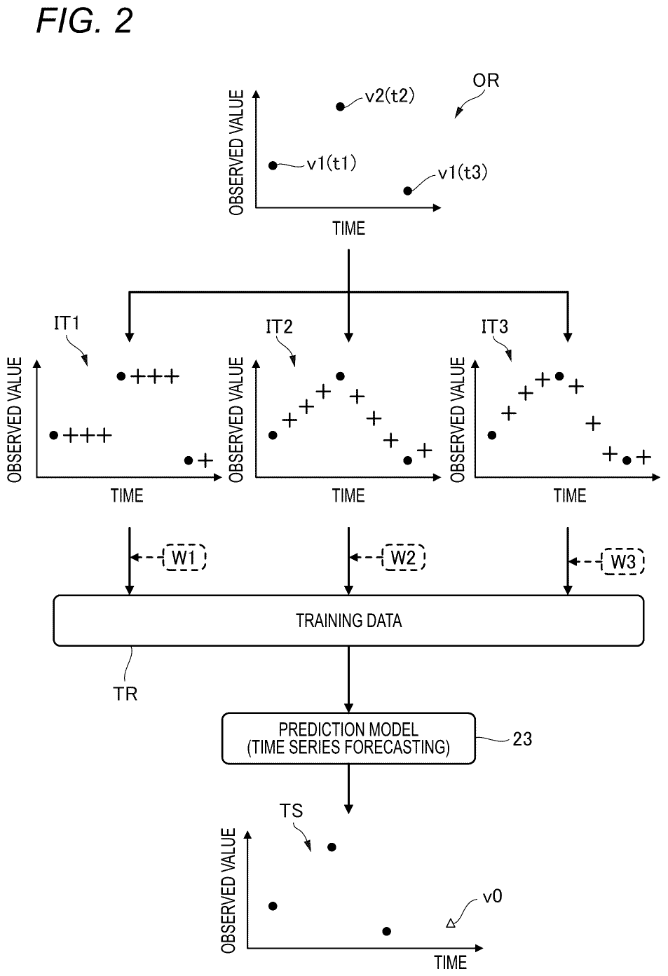

A method of generating the training data TR that is executed by the processor 52 will be described with reference to FIG. 2.

In the illustrated example, the original data set OR includes an observed value v1 of the axillary temperature acquired at a time point t1, an observed value v2 of the axillary temperature acquired at a time point t2, and an observed value v3 of the axillary temperature acquired at the time point t3. The number of the multiple observed values included in the original data set OR may be arbitrarily determined.

The processor 52 is configured to generate multiple interpolation data sets in different manners based on the original data set OR. In this example, a first interpolation data set IT1, a second interpolation data set IT2, and a third interpolation data set IT3 are generated.

In the first interpolation data set IT1, three values of the axillary temperatures are interpolated in a time period between the time points t1 and t2, as well as in a time period between the time points t2 and t3, respectively. The interpolation method is based on the LOCF (Last Observation Carried Forward) method. In this method, interpolation is performed such that an observed value at a certain time point is maintained until a next observed value is obtained.

In the second interpolation data set IT2, three values of the axillary temperatures are interpolated in a time period between the time points t1 and t2, as well as in a time period between the time points t2 and t3, respectively. The interpolation method is based on a linear interpolation method. In this method, a value corresponding to an observed value at an arbitrary time point that is located on a linear line connecting two observed values is interpolated.

In the third interpolation data set IT3, three values of the axillary temperatures are interpolated in a time period between the time points t1 and t2, as well as in a time period between the time points t2 and t3, respectively. The interpolation method is based on a spline interpolation method. In this method, a value corresponding to an observed value at an arbitrary time point that is located on a polynomial curve connecting two observed values is interpolated.

The processor 52 generates training data TR so as to include the first interpolation data set IT1, the second interpolation data set IT2, and the third interpolation data set IT3, and outputs the training data TR from the output interface 53.

As long as the interpolation methods are different from each other, the number of the multiple interpolation data sets to be generated by the processor 52 may be arbitrarily determined. An arbitrary interpolation data set selected from the multiple interpolation data sets as generated may be an example of the first interpolation data set. Similarly, any other interpolation data set selected from the multiple interpolation data sets may be an example of the second interpolation data set.

It should be noted that, as used herein, the expression “different interpolation methods” is not intended to refer to only a case where the types of interpolation methods are different from each other. As an example, an interpolation data set that is generated by spline interpolation using a cubic polynomial, and an interpolation data set that is generated by spline interpolation using a fifth polynomial are interpreted as having the same type of [spline interpolation], but having different interpolation methods. As another example, multiple interpolation data sets generated with the same type of interpolation method but the number of values to be interpolated during the same time period are different from each other are interpreted as “different interpolation methods” as well.

In general, the larger the amount of data included in the training data TR (in this example, the number of observed values), the more the capability of suppressing degradation of accuracy of the prediction performed by the prediction model 23 to be generated. On the other hand, there may be a case where it is difficult to ensure time series data of a certain observed parameter including a sufficient number of observed values, like the axillary temperature in this example. However, according to the configuration of the present exemplary embodiment, the amount of data included in the training data TR through the interpolation processing can be augmented even in the case where the number of observed values included in the original data set OR is small. In addition, since the training data TR is generated so as to include multiple interpolation data sets generated with different interpolation methods, it is possible to suppress a bias in the augmentation tendency that may be caused by relying on a specific interpolation method. Accordingly, it is possible to suppress degradation of prediction accuracy of a prediction model adapted to predict physiological information even in an environment in which it is difficult to acquire a sufficient amount of time series data of an observed parameter.

Particularly in the present exemplary embodiment, multiple interpolation data sets are generated with different types of interpolation methods. Accordingly, the above-described bias of the augmentation tendency can be further suppressed.

The processor 52 may be configured to assign a weighting factor to each of the multiple interpolation data sets as generated. The weighting factor corresponds to the impact on the machine learning. In other words, in a case where the data amounts included in the two interpolation data sets are the same, the interpolation data set to which a larger weighting factor is assigned has a greater impact on the machine learning.

In the present exemplary embodiment, the first weighting factor w1 is assigned to the first interpolation data set IT1. Similarly, a second weighting factor w2 is assigned to the second interpolation data set IT2, and a third weighting factor w3 is assigned to the third interpolation data set IT3. In this case, the processor 52 is configured to provide at least two kinds of weighting factors. The first weighting factor w1 may be different from each of the second weighting factor w2 and the third weighting factor w3, or the same as either the second weighting factor w2 or the third weighting factor w3. In other words, the multiple interpolation data sets include at least two interpolation data sets to which different weighting factors are assigned.

The adjustment of the weighting factor may be performed by adjusting a value of loss in a loss function that is used in the machine learning, or may be performed by adjusting the amount of data that is finally included in the training data TR. For example, in a case where it is known in advance that the spline interpolation is more accurate than the LOCF interpolation (the error from the measured value is small), adjustment may be performed such that the amount of data included in the training data TR from the third interpolation data set IT3 is made larger than the amount of data included in the training data TR from the first interpolation data set IT1.

According to such a configuration, it is possible to realize machine learning in which different accuracies between the different interpolation methods have been considered. As a result, it is possible to reduce an influence on the prediction accuracy of the prediction model 23 that is generated through the use of an interpolation method with relatively low accuracy.

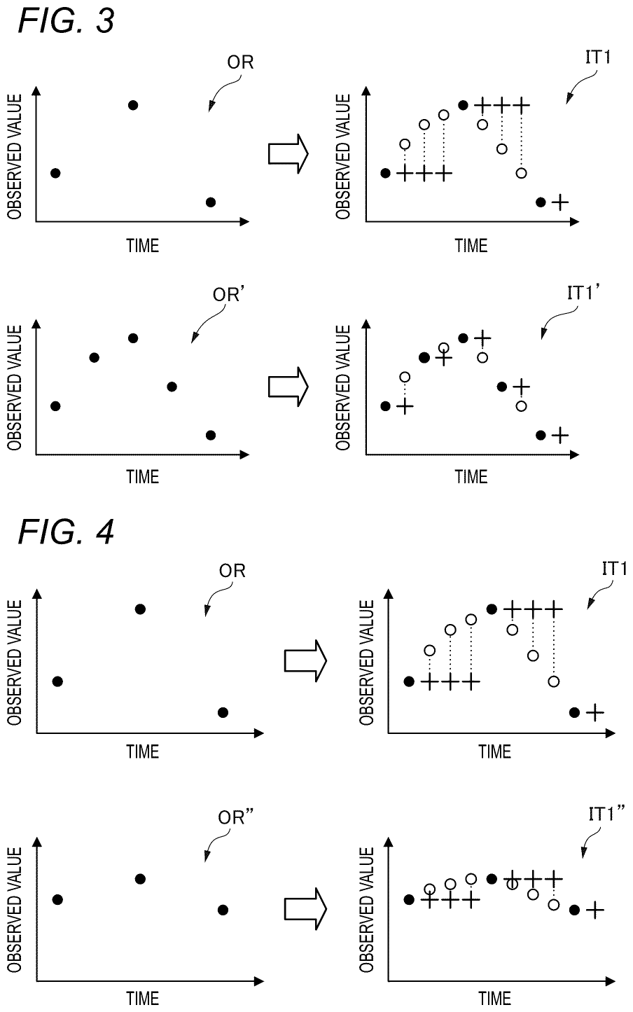

FIG. 3 illustrates another exemplary way for assigning the weighting factor that is executed by the processor 52. In this example, the weighting factor is changed in accordance with the number of observed values per unit time included in the original data set OR.

In FIG. 3, a first interpolation data set IT1′ using LOCF interpolation is generated based on the original data set OR. The white circles indicated in the first interpolation data set IT1 represent true values of the observed values. Namely, the length of each dashed line extending in the vertical direction corresponds to an amount of error of an interpolated value with respect to the true value.

On the other hand, a first interpolation data set IT1′ using the LOCF interpolation is generated based on the original data set OR′. The number of observed values per unit time included in the original data set OR′ is larger than the number of observed values per unit time of the original data set OR. The error of the interpolated value with respect to the true value in the first interpolation data set IT1′ is less than that of the first interpolation data set IT1.

The processor 52 may be configured to decrease a weighting factor to be assigned to the interpolation data set generated through the use of the LOCF interpolation as the less the number of observed values per unit time included in the original data set OR. On the other hand, the processor 52 may be configured to increase a weighting factor to be assigned to the interpolation data set generated through the use of the linear interpolation or the spline interpolation with relatively high accuracy.

For example, in a case where a possible narrowest time interval for acquiring the observed values is denoted by ΔT, and an actual time interval for acquiring the observed values is denoted by Δt, the processor 52 may determine, based on the following equations, a weighting factor W_{L} that is to be assigned to the interpolation data set generated through the use of the LOCF interpolation and a weighting factor w_{S} that is to be assigned to the interpolation data set generated through the use of the spline interpolation.

$$\begin{matrix}{w}_{L} = Δ T / Δ t \\ {w}_{S} = 1 - ( Δ T / Δ t )\end{matrix}$$

According to such a configuration, it is possible to suppress such an impact on the machine learning that degrades the prediction accuracy of the prediction model 23 that is caused by an interpolation data set generated through the use of an interpolation method with relatively low accuracy with respect to an original data set OR provided with a smaller number of observed values.

FIG. 4 illustrates another exemplary way for assigning the weighting factor that is executed by the processor 52. In this example, the weighting factor is changed in accordance with the variation width of observed values included in the original data set OR. Also in FIG. 4, a first interpolation data set IT1 using the LOCF interpolation is

generated based on the original data set OR. The white circles indicated in the first interpolation data set IT1 represent true values of the observed values. Namely, the length of each dashed line extending in the vertical direction corresponds to an amount of error of an interpolated value with respect to the true value.

On the other hand, a first interpolation data set IT1″ using the LOCF interpolation is generated based on the original data set OR″. The variation width of observed values included in the original data set OR″ is larger than the variation width of observed values included in the original data set OR. The error of the interpolated value with respect to the true value in the first interpolation data set IT1″ is less than that of the first interpolation data set IT1.

The processor 52 may be configured to decrease a weighting factor to be assigned to the interpolation data set generated through the use of the LOCF interpolation as the larger the variation width of observed values per unit time included in the original data set OR. On the other hand, the processor 52 may be configured to increase a weighting factor to be assigned to the interpolation data set generated through the use of the linear interpolation or the spline interpolation with relatively high accuracy.

According to such a configuration, it is possible to suppress such an impact on the machine learning that degrades the prediction accuracy of the prediction model 23 that is caused by an interpolation data set generated through the use of an interpolation method with relatively low accuracy with respect to an original data set OR in which the variation width of observed values is large.

The change of the weighting factor based on the variation width of the observed values may be performed with respect to the same observed parameter, or may be performed with respect to different types of observed parameters in a case where the prediction model 23 uses multiple observed parameters as inputs. For example, blood pressure value, respiration rate, heart rate, and the like have a relatively large variation width. On the other hand, body temperature, index values acquired through the blood examination, and the like generally have a relatively small variation width.

As another example, let p(w1, w2, w3) be the performance of the prediction model 23 that is generated by learning the training data TR generated with specific values of a first weighting factor w1, second weighting factor w2, and third weighting factor w3. Here, w1+w2+w3=1, and the initial allotment of weighting factors is set as w1=w2=w3=⅓.

The processor 42 calculates a value of (pᵢ′−p) based on the following definition of pᵢ′, and select the maximum one as a new allotment of the weighting factors. The above calculation is repeated until the value of (pᵢ′−p) falls below E. ¿ is a sufficiently small value. A is an arbitrary value representing a variation from an original weighting factor to a new weighting factor.

$$\begin{matrix}{p}_{1} ’ ( w 1 + Δ , w 2 - Δ / 2 , w 3 - Δ / 2 ) \\ {p}_{2} ’ ( w 1 - Δ / 2 , w 2 + Δ , w 3 - Δ / 2 ) \\ {p}_{3} ’ ( w 1 - Δ / 2 , w 2 - Δ / 2 , w 3 + Δ )\end{matrix}$$

As illustrated in FIG. 1, the prediction model generating device 4 includes an input interface 41. The input interface 41 is configured to receive the training data TR generated as described above.

The prediction model generating device 4 includes a processor 42 and an output interface 43. The processor 42 is configured to cause a neural network to learn the training data TR to generate the prediction model 23, and output the prediction model 23 from the output interface 43. The prediction model 23 outputted from the output interface 43 is installed in the prediction device 2.

In the case of the example illustrated in FIG. 2, the processor 42 executes supervised learning with the training data TR such that the observed value of the axillary temperature that is acquired after the time point t3 in the original data set OR is regarded as the ground truth. The prediction model 23 to be accordingly generated is configured to predict, in response to the input of the time series data TS for the axillary temperature (multiple observed values acquired from the subject S at different time points), an unobserved value v0 of the axillary temperature, as a time series forecasting model. The unobserved value v0 of the axillary temperature is an example of the physiological information.

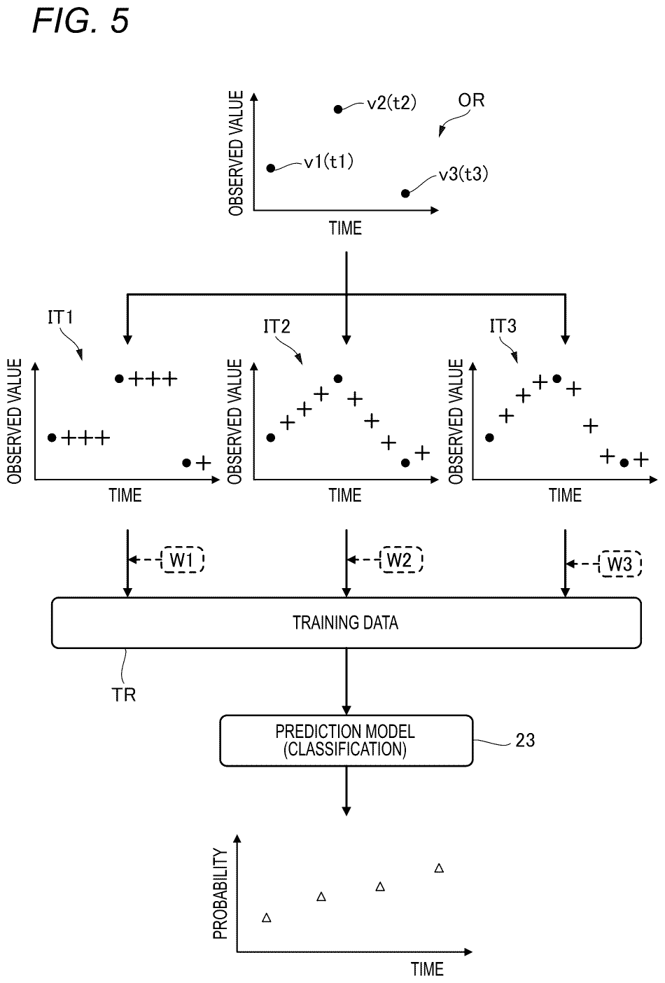

FIG. 5 illustrates another exemplary machine learning performed by the processor 42. The processor 42 executes supervised learning with the training data TR such that whether an event that requires an antipyretic analgesic agent occurred at or after the time point t3 is regarded as the ground truth. The event that requires an antipyretic analgesic agent is an example of an event related to the observed parameter. The prediction model 23 to be accordingly generated is configured to predict, in response to the input of the time series data TS for the axillary temperature (multiple observed values acquired from the subject S at different time points), a probability of occurrence of the event that requires an antipyretic analgesic agent, as a classification model. The probability of occurrence of the event that requires an antipyretic analgesic agent is an example of the physiological information.

As the probability of occurrence of an event related to the observed parameter, only a value at a specific time point may be predicted, or time series data including multiple predicted values may be formed as illustrated in FIG. 5. The predicted value of the probability of occurrence may or may not be subjected to visualization processing.

Other examples of the physiological information for which the probability of occurrence is predicted by the classification model may include: a probability of occurrence of an event involving respiratory management with a respirator with respect to an input of time series data including observed values of respiratory rate, heart rate, arterial oxygen saturation or the like that are intermittently observed; a probability of occurrence of an acute renal failure with respect to an input of time series data including urine volumes that are regularly measured; and a probability of death in hospital with respect to an input of time series data including observed values of respiratory rate, heart rate, blood pressure or the like that are intermittently observed.

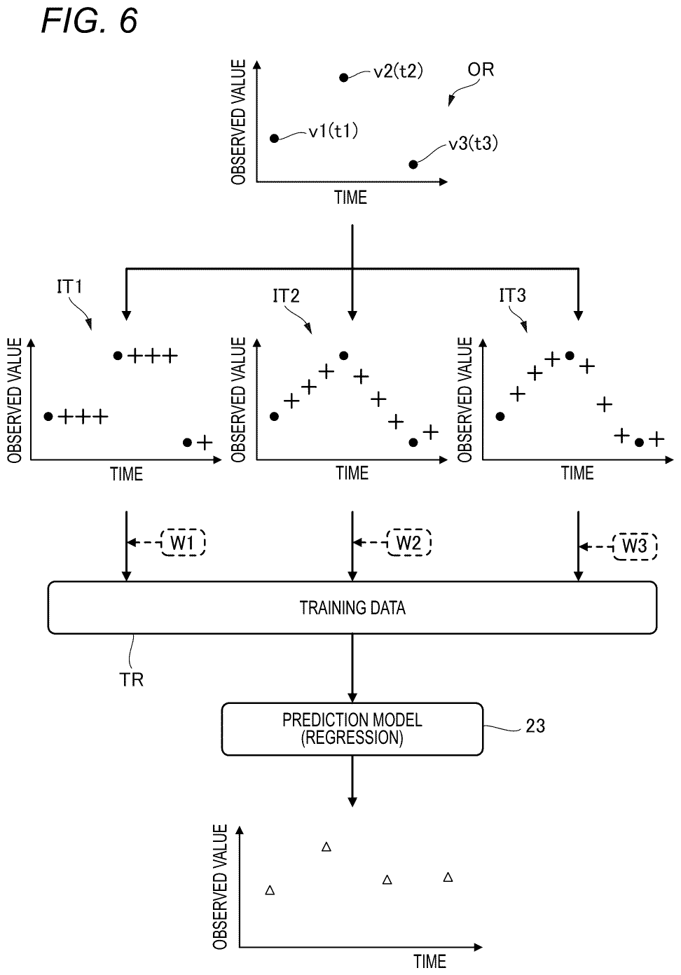

FIG. 6 illustrates another exemplary machine learning performed by the processor 42. The processor 42 executes supervised learning with the training data TR such that the observed value of deep body temperature that is acquired at or after the time point t3 in the original data set OR is regarded as the ground truth. The deep body temperature is an example of another observed parameter. The prediction model 23 to be accordingly generated is configured to predict, in response to the input of the time series data TS for the axillary temperature (multiple observed values acquired from the subject S at different time points), an observed value of the deep body temperature, as a regression model. The observed value of the deep body temperature is an example of the physiological information.

As the observed value of another observed parameter, only the value at a specific time point may be predicted, or time series data including multiple predicted values may be formed as illustrated in FIG. 6. The predicted observed value may or may not be subjected to visualization processing.

Other examples of the observed parameter for which the observed value is predicted with the regression model may include: a cardiac index with respect to an input of time series data including observed values of heart rate, non-invasive blood pressure or the like that are intermittently observed; a blood creatinine with respect to an input of time series data including urine volumes that are regularly measured; and a remaining length of stay in an intensive care unit (ICU) with respect to an input of blood examination results that are regularly performed as well as time series data including observed values of body temperature, respiratory rate, heart rate, blood pressure or the like that are intermittently observed.

As illustrated in FIG. 1, the prediction system 1 may include an evaluation system 6. The evaluation system 6 is configured to perform a performance evaluation of the prediction model 23 that is installed in the prediction device 2. As an example, the evaluation system 6 may include an adequate configuration adapted to receive feedback from the user with respect to the prediction result outputted by the prediction model 23. The feedback may take the form of a score or a comment.

The input interface 51 of the training data generating device 5 may be adapted to receive feedback data FB. The feedback data FB includes, as information, a breakdown of weighting factors employed in a prediction model 23 that has obtained a high evaluation through the evaluation system 6.

According to such a configuration, it is possible to generate a prediction model 23 with a higher adaptability to the user's environment, through re-learning based on the evaluation of the prediction model 23 that is installed in the prediction device 2 and is actually used.

Each of the processor 22 of the prediction device 2, the processor 42 of the prediction model generating device 4, and the processor 52 of the training data generating device 5 having various functions described above may be implemented by one or more non-exclusive microprocessors configured to cooperate with one or more non-exclusive memories. Examples of the non-exclusive microprocessor include a CPU, an MPU, and a GPU. Examples of the non-exclusive memory include a ROM and a RAM. In this case, a computer program for executing the above-described processing may be stored in the ROM. The ROM is an example of a non-transitory computer-readable medium having stored a computer program. The non-exclusive microprocessor designates at least a part of the program stored in the ROM, loads the designated program in the RAM, and executes the above-described processing in cooperation with the RAM. The computer program may be pre-installed in a non-exclusive memory, or may be downloaded from an external server with a communication network, and then installed in the non-exclusive memory. In this case, the external server is an example of a non-transitory computer readable medium having stored a computer program.

Each of the processor 22, the processor 42, and the processor 52 may be implemented by one or more exclusive integrated circuitries capable of executing the above-described computer program. Examples of the exclusive integrated circuitry include a microcontroller, an ASIC, and an FPGA. In this case, the above-described computer program is pre-installed in a memory element included in the exclusive integrated circuitry. The memory element is an example of a non-transitory computer-readable medium having stored a computer program. Each of the processor 22, the processor 42, and the processor 52 may also be implemented by a combination of the non-exclusive microprocessor and the exclusive integrated circuitry.

Each of the configurations exemplified above is merely illustrative for facilitating understanding of the presently disclosed subject matter. Each exemplary configuration may be appropriately modified or combined with another exemplary configuration within the scope of the presently disclosed subject matter.

In the above exemplary embodiment, the prediction model generating device 4 and the training data generating device 5 are illustrated as devices independent from each other. In this case, the input interface 21 and the output interface 43 are configured as hardware interfaces. However, both devices may be implemented as different functional modules in a single device. In this case, both interfaces may be implemented as software interfaces.

In the above exemplary embodiment, the prediction device 2 and the prediction model generating device 4 are illustrated as devices independent from each other. In this case, the input interface 21 and the output interface 43 are configured as hardware interfaces. However, both devices may be implemented as different functional modules in a single device. In this case, both interfaces may be implemented as software interfaces.

The prediction model 23 need not be installed in the prediction device 2. Although not illustrated, the prediction device 2 may be connected to an external server device such that communication with the external server device via a communication network is enabled. In this case, the prediction model 23 may be installed in the external server device.

In the above exemplary embodiment, the prediction model generating device 4 generates the prediction model 23 through the machine learning using the neural network. However, the prediction model 23 may be generated through other machine learning algorithms. Examples of other machine learning algorithms include linear regression, logistic regression, decision trees, random forests, support vector machines, and the like.

As used herein, the meaning of the term “prediction” may be interpreted so as not to exclude the meaning of each of the terms “estimation” and “forecasting”.

1. A method of generating, with a computing device, training data to be used in machine learning of a prediction model adapted to predict physiological information, comprising:
receiving an original data set including a first observed value of an observed parameter for obtaining the physiological information that is acquired from a living body at a first time point, and a second observed value of the observed parameter that is acquired from the living body at a second time point that is different from the first time point;
generating a first interpolation data set by interpolating, with a first method, at least one value of the observed parameter in a time period between the first time point and the second time point;
generating a second interpolation data set by interpolating, with a second method that is different from the first method, at least one value of the observed parameter in the time period; and
generating the training data so as to include the first interpolation data set and the second interpolation data set.

2. The method according to claim 1,
wherein the first method and the second method differ in a type of interpolating method.

3. The method according to claim 1,
wherein a weighting factor corresponding to an impact on the machine learning is assigned to each of the first interpolation data set and the second interpolation data set; and
wherein the weighting factor assigned to the first interpolation data set is different from the weighting factor assigned to the second interpolation data set.

4. The method according to claim 3,
wherein the weighting factor is changed in accordance with a number per unit time of observed values included in the original data set.

5. The method according to claim 3,
wherein the weighting factor is changed in accordance with a variation width of observed values included in the original data set.

6. The method according to claim 3,
wherein the weighting factor is changed in accordance with an evaluation result of performance of the prediction model.

7. A method of generating, with a computing device, the prediction model with the training data generated by the method according to claim 1, comprising:
performing supervised machine learning such that an observed value of the observed parameter that is acquired after the second time point is regarded as a ground truth; and
configuring the prediction model so as to predict, with respect to multiple observed values of the observed parameter acquired at different time points as an input, an unobserved value of the observed parameter as the physiological information.

8. A method of generating, with a computing device, the prediction model with the training data generated by the method according to claim 1, comprising:
performing supervised machine learning such that whether an event related to the observed parameter occurred at or after the second time point is regarded as a ground truth; and
configuring the prediction model so as to predict, with respect to multiple observed values of the observed parameter acquired at different time points as an input, a probability of occurrence of the event as the physiological information.

9. A method of generating, with a computing device, the prediction model with the training data generated by the method according to claim 1, comprising:
performing supervised machine learning such that an observed value of a different observed parameter from the observed parameter that is acquired after the second time point is regarded as a ground truth; and
configuring the prediction model so as to predict, with respect to multiple observed values of the observed parameter acquired at different time points as an input, an observed value of the different observed parameter as the physiological information.

10. A computing device configured to generate training data to be used in machine learning of a prediction model adapted to predict physiological information, comprising:
an interface configured to receive an original data set including a first observed value of an observed parameter for obtaining the physiological information that is acquired from a living body at a first time point, and a second observed value of the observed parameter that is acquired from the living body at a second time point that is different from the first time point; and
a processor configured to generate the training data so as to include a first interpolation data set and a second interpolation data set,
wherein the first interpolation data set is generated by interpolating, with a first method, at least one value of the observed parameter in a time period; and
wherein the second interpolation data set is generated by interpolating, with a second method that is different from the first method, at least one value of the observed parameter in the time period between the first time point and the second time point.

11. A non-transitory computer-readable medium having stored a computer program adapted to be executed by a processor installed in a computing device, the computer program being configured to cause, when executed, the computing device to:
receive an original data set including a first observed value of an observed parameter for obtaining the physiological information that is acquired from a living body at a first time point, and a second observed value of the observed parameter that is acquired from the living body at a second time point that is different from the first time point;
generate a first interpolation data set by interpolating, with a first method, at least one value of the observed parameter in a time period between the first time point and the second time point;
generate a second interpolation data set by interpolating, with a second method that is different from the first method, at least one value of the observed parameter in the time period; and
generate the training data so as to include the first interpolation data set and the second interpolation data set.
